(19) United States
(12) Patent Application Publication
(10) Pub. No.: US 2016/0310352 A1
(43) Pub. Date: Oct. 27, 2016

(54) INTERCHANGEABLE MASSAGE ROLLER

(71) Applicant: GYMWELL CO., LTD., TAINAN CITY (TW)

(72) Inventor: TIEN-MING CHEN, TAINAN CITY (TW)

(21) Appl. No.: 14/695,227

(22) Filed: Apr. 24, 2015

(51) Int. Cl. A61H 15/02; A61F 7/00

(52) U.S. Cl. CPC A61H 15/02; A61F 7/00

(57) ABSTRACT

An interchangeable massage roller is disclosed, which comprises a roller body having an annular depression around the middle section. The annular depression is selectively wrapped around by a cold/hot compress pad or a massage pad with different hardness and different projections according to the different need. Therefore, a cold/hot compress or a rolling massage course is applied to a user by the interchangeable massage roller according to the user's need, achieving the multifunction and selectable effect.

### BACKGROUND OF THE INVENTION

1. Field of the Invention

The present invention relates an interchangeable massage roller. More particularly, a pad wrapped around a massage roller is selectively interchangeable according to different massage or cold/hot compress courses.

2. Description of Related Art

As the rapidly development of industry and commerce, the modem life is busy and nervous. Office workers often sit in the office and are lack of exercise, so they usually suffer from neck and shoulder pain, waist and back stiff, or muscle rigidity. In order to try to keep body in the best situation to have the ability for overcoming challenge, people release their pressure not only through the suitable leisure activities, the suitable massage for releasing pressure is also popular.

In order to keep body in the best situation, people need timely exercise to keep the best ability and the best posture. When it comes to exercise, it is hard to avoid the problem of muscle injury resulted from excessive or improper exercise. When people suffer injury, a cold compress is generally applied onto the injured part to alleviate inflammation, after that, a hot compress is applied for promoting blood circulation. In general, massage is operated together with a hot compress. Or after exercise, muscle feels minor pain, and then it needs to release pain via massage operation.

However, the device for hot/cold compress is general independent of massage device, so the massage and the hot/cold compress are operated independently. In general, the compress pad is a sheet and is heated or cooled to apply to the injured part. But the sheet of the compress pad is not easily to fix on the injured part, it is inconvenient to to use. When a large part of injured body needs to hot/cold compress, the hot/cold compress pad is applied to one part of the injured body for a few minutes, and then the hot/cold compress pad is moved a little to the other part of the injured body for another few minutes to complete the whole hot/cold compress course. When the injured body needs to is massage, it needs to buy other massage device for releasing muscle pain.

Therefore, the traditional massage device and hot/cold compress pad are limited in usage and are inconvenient.

### SUMMARY OF THE INVENTION

Therefore, the object of the present invention is to provide an interchangeable massage roller. More particularly, a pad wrapped around a massage roller is selectively interchangeable according to different massage or cold/hot compress courses.

For the above object, an interchangeable massage roller is disclosed, which comprises a roller body having an annular depression around the middle section. The annular depression is selectively wrapped around by a cold/hot compress pad or a massage pad with different hardness and different projections according to the different need. Therefore, a cold/hot compress or a rolling massage course is applied to a user by the interchangeable massage roller according to the user's need, achieving the multifunction and selectable effect.

According to an embodiment, the ends of the cold/hot compress pad comprises hooks and loops respectively to fasten each other for wrapping the cold/hot compress around the annular depression of the roller body.

According to an embodiment, the ends of the massage pad comprises hooks and loops respectively to fasten each other for wrapping the massage pad around the annular depression of the roller body.

According to an embodiment, the roller body further comprises two massage parts with concave-convex pattern beside two sides of the annular depression respectively.

### BRIEF DESCRIPTION OF THE DRAWINGS

FIG. 1 is an exploded view of an interchangeable massage roller according to an embodiment of the present invention;

FIG. 2 is an exploded view of an interchangeable massage roller according to another embodiment of the present invention;

FIG. 3 is an exploded view of an interchangeable massage roller according to the other embodiment of the present invention; and

FIG. 4 is a partial sectional view of an interchangeable massage roller according to the embodiment of the present invention.

### DETAILED DESCRIPTION OF THE PREFERRED EMBODIMENT

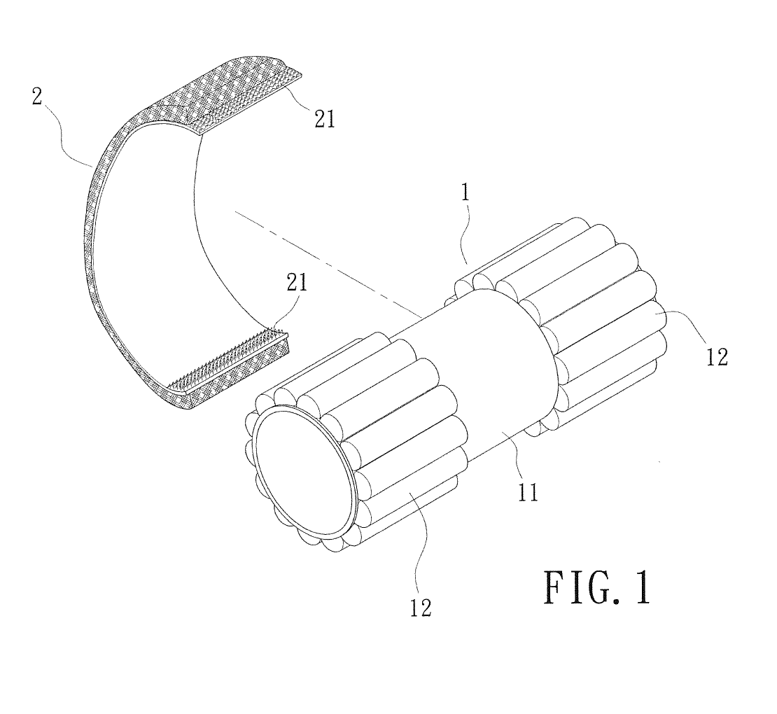

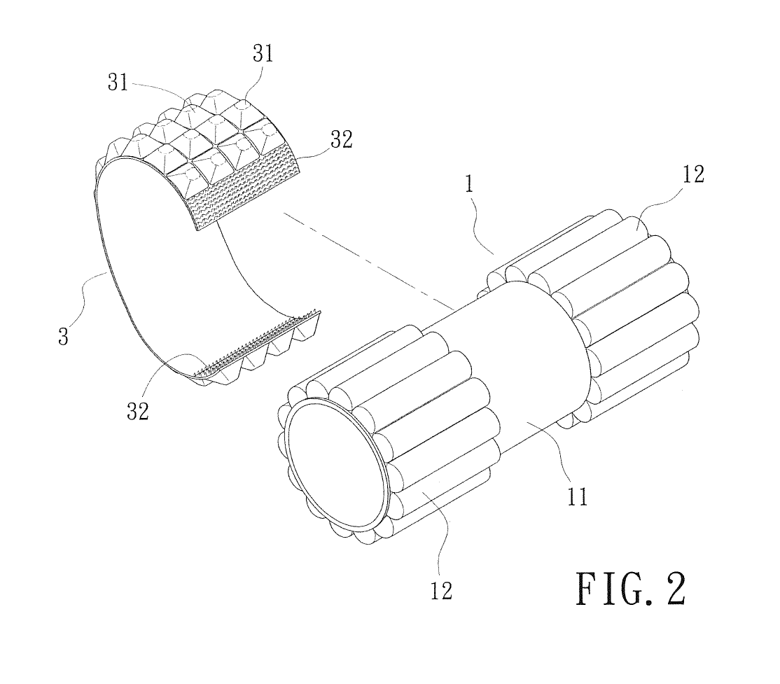

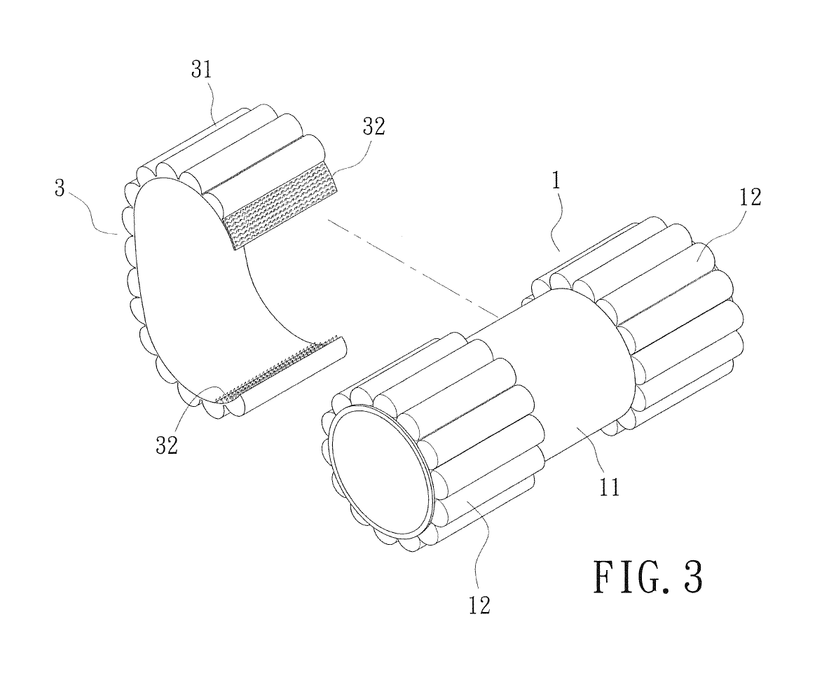

First, please refer to FIG. 1 to FIG. 3, which are exploded views of an interchangeable massage roller according to several different embodiments of the present invention. The interchangeable massage roller comprises a roller body 1, a cold/hot compress pad 2, and at least one massage pad 3.

The roller body 1 comprises an annular depression 11 around the middle section and two massage parts 12 with concave-convex pattern beside two sides of the annular depression 11 respectively. The roller body 1 is hollow or solid according to the need.

The cold/hot compress pad 2 is selectively wrapped around the annular depression 11 of the roller body 1.

The massage pad 3 is selectively wrapped around the annular depression 11 of the roller body 1, wherein the surface of the massage pad 3 comprises a plurality of projections 31.

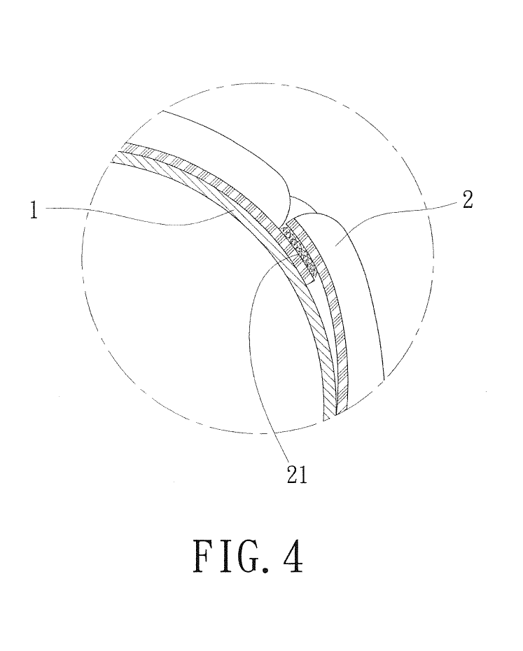

Please refer to FIG. 1 to FIG. 4. When the interchangeable massage roller 1 is used, an applicable pad is selected to wrap around the annular depression of the roller body according to the user's need. For example, when a cold/hot compress is given to a user, the cold/hot compress pad 2 is selected to wrap around the annular depression 11. Or, when a massage course is given to a user, the massage pad 3 is selected to wrap around the annular depression 11. When the interchangeable massage roller 1 rolls, the user's body is massaged by the projections 31. Furthermore, several kinds of the massage pads could be selected, for is example, the projections 31 of the massage pad 3 have different shape such as granules or strips, or the hardness of the projections 31 of the massage pads 3 are different. Moreover, the massage pad 3 has one or more than one kinds of the projections 31.

Please refer to FIG. 1 to FIG. 4. When a cold/hot compress is given to a user, the cold/hot compress pad 2 is selected to wrap around the annular depression 11. The two ends of the cold/hot compress pad 2 comprises hooks and loops 21 respectively to fasten each other for wrapping the cold/hot compress pad 2 around the annular depression 11 of the roller body 1. After the cold/hot compress pad 2 is wrapped around the annular depression 11, the cold/hot compress pad 2 rolls on the user's body which is compressed via the rolling of the interchangeable massage roller 1 to extend the compressed area, achieving the massage effect simultaneously.

Or, when a massage pad is selected, the fasten method of the massage pad 3 is the same as the cold/hot compress pad 2. The two ends of the massage pad 3 comprises hooks and loops 32 respectively to fasten each other for wrapping the massage pad 3 around the annular depression 11 of the roller body 1. The foregoing fasten method of the compress pad 2 and the massage pad 3 is an embodiment of the present invention, the other fasten methods for wrapping the compress pad 2 around the annular depression 11 of the roller body 1, such as interlock method, engagement method, or tighten method, may be made without departing from the spirit and the scope of the present invention.

According to the above description and embodiments, the interchangeable massage roller of the present invention has the advantages as following:

1. In the interchangeable massage roller of the present invention, an additional compress pad or massage pad is selected to wrap around the roller body, so the interchangeable massage roller is used to apply a massage with different level or a massage with a cold compress or a hot compress according to the user's need.

2. The compress pad wrapped around the roller body has the function of hot compress or cold compress, thereby advancing the effect of the interchangeable massage roller of the present invention.

3. In the interchangeable massage roller of the present invention, the compress pad and the massage pad are firmly wrapped around the roller body, so it is not loosen during rolling, achieving the effect of stable positioning.

What is claimed is:

1. An interchangeable massage roller, comprising in combination:
a roller body, comprising an annular depression around the middle section;
a cold/hot compress pad, selectively wrapping around the annular depression of the roller body; and
at least one massage pad, selectively wrapping around the annular depression of the roller body, wherein the surface of the massage pad comprises a plurality of projections.

2. The interchangeable massage roller according to claim 1, wherein the two ends of the cold/hot compress pad comprises hooks and loops respectively to fasten each other for wrapping the cold/hot compress around the annular depression of the roller body.

3. The interchangeable massage roller according to claim 1, wherein the two ends of the massage pad comprises hooks and loops respectively to fasten each other for wrapping the massage pad around the annular depression of the roller body.

4. The interchangeable massage roller according to claim 1, the roller body further comprising:
two massage parts with concave-convex pattern beside two sides of the annular depression respectively.

5. The interchangeable massage roller according to claim 1, wherein the projections of the massage pad are granules or strips.

6. The interchangeable massage roller according to claim 1, wherein the roller body is hollow or solid.

7. The interchangeable massage roller according to claim 2, further comprising:
two massage parts with concave-convex pattern beside two sides of the annular depression of the roller body respectively.

8. The interchangeable massage roller according to claim 2, wherein the projections of the massage pad are granules or strips.

9. The interchangeable massage roller according to claim 2, wherein the roller body is hollow or solid.

10. The interchangeable massage roller according to claim 3, further comprising:
two massage parts with concave-convex pattern beside two sides of the annular depression of the roller body respectively.

11. The interchangeable massage roller according to claim 3, wherein the projections of the massage pad are granules or strips.

12. The interchangeable massage roller according to claim 3, wherein the roller body is hollow or solid.
